(19) United States
(12) Patent Application Publication
(10) Pub. No.: US 2022/0372694 A1
(43) Pub. Date: Nov. 24, 2022

(54) ELASTIC FIBER AND FIBER STRUCTURE COMPRISING THE SAME

(71) Applicant: TORAY OPELONTEX CO., LTD, Osaka (JP)

(72) Inventors: Katsuya SUZUKI, Shiga (JP); Toshihiro TANAKA, Shiga (JP); Kazuki NAESHIRO, Shiga (JP); Keiichiro OSHIMA, Shiga (JP); Yasunobu ARAKAWA, Shiga (JP)

(21) Appl. No.: 17/770,141

(22) PCT Filed: Oct. 20, 2020
(86) PCT No.: PCT/IB2020/059855

§ 371 Date: Apr. 19, 2022

(30) Foreign Application Priority Data
Oct. 21, 2019 (JP) 2019-192070

(51) Int. Cl. D06M 15/227; A61L 15/26; D06M 13/02; D06M 15/233; D01F 1/10; D06M 15/643; D06M 13/144; D06M 13/188; D01F 6/70; D01F 11/08; D01D 5/04; D01D 11/06

(52) U.S. Cl. CPC D06M 15/227; A61L 15/26; D06M 13/02; D06M 15/233; D01F 1/106; D06M 15/643; D06M 13/144; D06M 13/188; D01F 6/70; D01F 11/08; D01D 5/04; D01D 11/06; D06M 2101/38

(57) ABSTRACT

An elastic fiber of the present invention having an elastic fiber treatment agent attached to the fiber surface, comprises: a hydrocarbon resin (A) having a structure in which a polymer including a structural unit whose monomer is at least one selected from aromatic olefins and aliphatic diolefins as the main structural unit is partially or fully hydrogenated; and a hydrocarbon oil (B). In this way, an elastic fiber and a fiber structure comprising the same are provided that are suitable for obtaining an elastic sheet that has excellent elastic fiber unraveling properties and adhesiveness to hot melt adhesives, and that exhibits good adhesiveness even when processed at a high draft, and for obtaining a sanitary product that is soft to the touch.

### TECHNICAL FIELD

The present invention relates to an elastic fiber having improved elastic fiber unraveling properties and excellent adhesiveness to hot melt adhesives.

### BACKGROUND ART

Elastic fibers are used in elastic clothing applications such as legwear, innerwear, and sportswear because of their excellent elastic properties. In recent years, large amounts of these elastic fibers have been used in sanitary applications (sanitary materials) such as disposable diapers and sanitary napkins. Disposable sanitary products such as disposable diapers and sanitary napkins need to be stretchable in order to provide the wearer with improved fit. Disposable diapers in particular have been devised in various ways to allow for expansion and contraction around the waist, legs, and torso. Use of a woven fabric (stretch fabric) with elasticity in the material itself has been considered, but it is too expensive for use in disposable products. Therefore, usually, yarn-like or band-like stretchable members are attached in an elongated state to non-stretchable members such as non-woven fabric or plastic film to make the non-stretchable members stretchable and form elastic sheets and gathers (see, for example, Patent Document 1). Specifically, strip-like rubber cords and yarn-like polyurethane elastic fibers are used as members fastened to the non-stretchable members to impart elasticity, and a hot melt adhesive is used for bonding. The use of various additives in polyurethane elastic fibers for improving the hot melt adhesiveness is disclosed in Patent Document 2. The application of an oil agent to impart both unraveling properties and hot melt adhesiveness to polyurethane elastic yarns is disclosed in Patent Document 3.

### PRIOR ART DOCUMENTS

### Patent Documents

[Patent Document 1] JP 2002-035029 A
[Patent Document 2] JP 2010-168717 A
[Patent Document 3] WO 2016/143499 A1

### DISCLOSURE OF THE INVENTION

### Problem to be Solved by the Invention

When a conventional elastic fiber used to impart elasticity such as the elastic fiber in Patent Document 1 is drafted up and attached, resistance from the elastic fiber is high when stretched, and yarn may get pulled out. When a larger amount of hot melt adhesive is used to avoid this, instead of reducing yarn pull-out, the finish of the members is hard, and the elasticity of the product as a whole is unsatisfactory. When the technique in Patent Document 2 is applied and additives are used in an attempt to improve hot melt adhesiveness, the unraveling properties of the elastic fiber deteriorate, and yarn breakage is more likely to occur during production of the elastic members. Patent Document 3 also requires further improvement in hot melt adhesiveness.

It is an object of the present invention to solve these problems associated with the prior art by providing an elastic fiber and a fiber structure comprising the same are provided that are suitable for obtaining an elastic sheet that has excellent elastic fiber unraveling properties and adhesiveness to hot melt adhesives, and that exhibits good adhesiveness even when processed at a high draft, and for obtaining a sanitary product that is soft to the touch.

### Means for Solving the Problem

The present invention is an elastic fiber having an elastic fiber treatment agent attached to the fiber surface, comprising: a hydrocarbon resin (A) having a structure in which a polymer including a structural unit whose monomer is at least one selected from aromatic olefins and aliphatic diolefins as the main structural unit is partially or fully hydrogenated; and a hydrocarbon oil (B). The present invention is also a fiber structure comprising this elastic fiber.

### Effects of the Invention

The present invention is able to provide an elastic fiber and a fiber structure comprising the same in which the elastic fiber has stable unraveling properties and good adhesiveness to hot melt adhesives when a hot melt adhesive is used. Because the elastic properties of elastic fibers are not impaired, an elastic sheet exhibiting good adhesiveness and low stress stretchability can be obtained even when the elastic fiber is processed at a high draft. Sanitary products such as disposable diapers and sanitary napkins can be manufactured without yarn breakage even when the manufacturing speed is increased, and costs can be reduced by reducing the amount of hot melt adhesive. The hot melt adhesive retention rate can be evaluated as an indicator of adhesiveness. In a sanitary product requiring less hot melt adhesive, the members are less hard due to the reduction in hot melt adhesive and the texture is softer. As a result, comfort and fit are excellent.

### BRIEF DESCRIPTION OF THE DRAWINGS

FIG. 1 is a schematic diagram used to explain the elastic fiber unraveling stability tester used in the examples of the present invention.

FIG. 2A and FIG. 2B are schematic diagrams used to explain the hot melt adhesiveness testing method in the same.

### MODE FOR CARRYING OUT THE INVENTION

The following is a detailed description of the present invention. There are no particular restrictions on the hydrocarbon resin (A) in the present invention as long as it has a structure in which a polymer including a structural unit whose monomer is at least one selected from aromatic olefins and aliphatic diolefins as the main structural unit is partially hydrogenated (sometimes referred to as partial hydrogenation below) and/or fully hydrogenated (sometimes referred to as full hydrogenation below). In the present invention, partial hydrogenation usually means at least 50% and less than 100% of the double bonds in the polymer are hydrogenated. When “hydrogenation” is used alone, it indicates a range including both partial hydrogenation and full hydrogenation. In the present specification, a “polymer including a structural unit whose monomer is an aromatic olefin and/or aliphatic diolefin as the main structural unit” is referred to as a “hydrocarbon resin precursor polymer.” In general, “hydrocarbon resin precursor polymer!” and “hydrocarbon resin (A)” are referred to simply as “petroleum resins” and are often indistinguishable. In the present invention, this distinction shall be made depending on the structure. A completely hydrogenated “hydrocarbon resin (A)” may be referred to as a saturated hydrocarbon resin. A hydrocarbon resin (A) may have different types of structural units and partially hydrogenated structures, and it is sometimes difficult to accurately express the structure using a chemical name. Therefore, for the sake of convenience, the structure of the monomer prior to hydrogenation is specified in the following description. In other words, when a monomer is described, the structure from which it is derived is specified, and the raw materials are not limited. In the present invention, “the main structural unit” refers to a hydrocarbon structural unit whose monomer is at least one selected from aromatic olefins and aliphatic diolefins that constitutes 90% by mass or more of the polymer.

In the present invention, the hydrocarbon resin (A) preferably has a structure in which the polymer containing a structural unit with an aromatic olefin as the monomer is partially or completely hydrogenated, and the aromatic olefin is indene and/or methylstyrene. In this way, a treatment agent can be provided for elastic fibers with good unraveling properties and adhesiveness when a hot melt adhesive is used.

Also, the hydrocarbon resin (A) preferably has a structure in which the polymer containing a structural unit with an aliphatic diolefin as the monomer is partially or completely hydrogenated, and the aliphatic diolefin is isoprene (including optical isomers).

The softening point of the hydrocarbon resin (A) is preferably 70° C. or higher and 140° C. or lower. In this way, heat softening occurs below the bonding temperature of hot melt adhesive, and a treatment agent can be provided for elastic fibers with good adhesiveness when a hot melt adhesive is used.

The elastic fiber comprises 0.1% by mass or more and 40% by mass or less, more preferably 1 to 20% by mass, and even more preferably 3 to 10% by mass of hydrocarbon resin (A) when the treatment agent is used as a parameter. This results in better affinity with the hot melt adhesive.

Also, 10% by mass or more of the hydrocarbon resin (A) preferably dissolves in the hydrocarbon oil (B) at 20° C., and the hydrocarbon resin is preferably insoluble in N,N-dimethylacetamide (DMAc) and/or N,N-dimethylformamide (DMF).

The swelling rate of polyurethane is 2.5% or less, preferably 2.2% or less, and more preferably 2.0% or less, when the treatment agent is attached to the polyurethane. This keeps the hydrocarbon oil (B) from impregnating the polyurethane elastic fiber, and a stable fiber morphology can be maintained.

The petroleum resins serving as hydrocarbon resin precursor polymers and hydrocarbon resins (A) include “C9-based petroleum resins” whose monomers are mainly aromatic olefins, “C5-based petroleum resins” whose monomers are mainly aliphatic diolefins, and “C5/C9-based petroleum resins” that are a mixture of these. Here, “whose monomers are mainly aromatic olefins” means structural units derived from aromatic olefins constituting more than 50 mol % of the whole, including structural units derived from other monomers. Similarly, “whose monomers are mainly aliphatic diolefins” means structural units derived from aliphatic diolefins constituting more than 50 mol % of the whole, including structural units derived from other monomers.

Alkylbenzenes and aromatic olefins are the main components of monomers that provide structural units to C9-based petroleum resins (sometimes referred to below as C9-based petroleum resin monomers). Examples of alkylbenzenes include isopropylbenzene, n-propylbenzene, 1-methyl-2-ethylbenzene, 1-methyl-3-ethyl benzene, 1-methyl-4-ethylbenzene, 1,3,5-trimethylbenzene, 1,2,3-trimethylbenzene, 1,2,4-trimethylbenzene, 1-methyl-2-n-propylbenzene, 1-methyl-3-n-propylbenzene, 1-methyl-4-isopropylbenzene, 1,3-diethylbenzene, and 1,4-diethylbenzene.

Examples of aromatic olefins include α-methylstyrene, 3-methylstyrene, o-methylstyrene, m-methylstyrene, p-methylstyrene, indene, m-methylpropenylbenzene, m-methylisopropenylbenzene, p-methylisopropenylbenzene, o-ethylstyrene, m-ethylstyrene, p-ethylstyrene, m,m-dimethylstyrene, dimethyl styrene, and methyl indene. When a hydrocarbon resin precursor polymer or hydrocarbon resin (A) in the present invention contains a C9-based petroleum resin, indene and methylstyrene are preferably included as monomers.

Examples of monomers that provide structural units to C5-based petroleum resins (sometimes referred to below as C5-based petroleum resin monomers) include 1-pentene, 2-pentene, 2-methyl-1 butene, 2-methyl-2-butene, cyclopentene, 1,3-pentadiene, isoprene, cyclopentadiene, and dicyclopentadiene. When a hydrocarbon resin precursor polymer or hydrocarbon resin (A) in the present invention contains a C5-based petroleum resin, isoprene is preferably included as a monomer.

When such a hydrocarbon resin (A) is included, the hot melt adhesiveness of elastic fibers can be improved in particular. Hydrogenated petroleum resins (C5-based petroleum resins and/or C9-based petroleum resins) have excellent compatibility with hydrocarbon oil (B) in the present invention and can be applied stably to the elastic fibers.

The softening point of the hydrocarbon resin (A) in the present invention is preferably 70° C. or higher and 140° C. or lower because this improves adhesiveness to hot melt adhesives. When a hydrocarbon resin (A) with a softening point of 70° C. or higher is used, the bonding strength to the hot melt adhesive is better in high temperature environments after the hot melt adhesive has been cured, and the creep resistance is also better. When a hydrocarbon resin (A) with a softening point of 140° C. or lower is used, compatibility with the hydrocarbon oil (B) is excellent during the manufacturing process described below. As a result, the hydrocarbon resin (A) can be dissolved in the hydrocarbon oil (B) at a high concentration, and the treatment agent can be easily adjusted. The softening point of the hydrocarbon resin (A) is measured in accordance with JIS K2207:2006.

Commercially available petroleum resin products that can be used as hydrocarbon resin (A) are commercially available hydrogenated and saturated hydrocarbon resin products. Examples include products which have the following structural components and have a softening point in the range from 70° C. to 140° C.
Partially hydrogenated petroleum hydrocarbon resins that are copolymerized petroleum resins with aliphatic components and aromatic components
Fully hydrogenated petroleum hydrocarbon resins that are copolymerized petroleum resins with aliphatic components and aromatic components
Fully hydrogenated petroleum hydrocarbon resins from aliphatic petroleum hydrocarbon resins
Partially hydrogenated petroleum hydrocarbon resins from aromatic petroleum hydrocarbon resins
Fully hydrogenated petroleum hydrocarbon resins from aromatic petroleum hydrocarbon resins

In the present invention, 10% by mass or more of the hydrocarbon resin (A) preferably dissolves in the hydrocarbon oil (B) at 20° C. When the hydrocarbon resin (A) has this solubility, the treatment agent can be easily adjusted, and elastic fibers with excellent hot melt adhesiveness and unraveling properties can be obtained. When 10% by mass or more of the hydrocarbon resin (A) dissolves in the hydrocarbon oil (B) at 20° C., it also has better affinity with hot melt adhesives.

There are no particular restrictions on the hydrocarbon oil (B) in the present invention as long as the content ratio of hydrocarbons with 6 to 60 carbon atoms is 90% or more and the hydrocarbon oil has fluidity at 30° C. There are also no particular restrictions on the chemical structure, which may be linear or branched. It may also include some hydroxyl groups as long as its hydrophobicity is not impaired. The hydrocarbon oil (B) is preferably a mineral oil from the standpoint of availability and cost.

Examples of mineral oils include aromatic hydrocarbons, paraffin hydrocarbons, and naphthenic hydrocarbons. One or more types can be used. The viscosity of the mineral oil at 40° C. using a Redwood viscometer is preferably from 30 seconds to 350 seconds, more preferably from 35 seconds to 200 seconds, and even more preferably from 40 seconds to 150 seconds. A paraffinic hydrocarbon is preferred as the mineral oil because it produces less odor.

If necessary, an elastic fiber treatment agent of the present invention may include a silicone oil (c), a higher alcohol (d), and a metal soap (e). There are no particular restrictions on the silicone oil (c). However, polydimethylsiloxane consisting of dimethylsiloxane units, polydialkylsiloxanes consisting of dimethylsiloxane units and dialkylsiloxane units containing alkyl groups with 2 to 4 carbon atoms, and polysiloxanes consisting of dimethylsiloxane units and methylphenylsiloxane units are preferably used. From the viewpoint of handling and reduction of running friction with guides, the viscosity at 25° C. is preferably from 5×10⁻⁶ to 50×10⁻⁶ m²/s. The viscosities are measured using the methods described in JIS-K 2283 (Crude Petroleum and Petroleum Products—Determination of Kinematic Viscosity and Calculation of Viscosity Index from Kinematic Viscosity). There are no particular restrictions on higher alcohols (d). Examples include linear and/or branched monoalcohols with 6 or more carbon atoms. Specific examples include linear alcohols such as hexanol, heptanol, octanol, nonaol, decanol, undecanol, 1-dodecanol, tridecanol, tetradecanol, pentadecanol, hexadecanol, heptadecanol, octadecanol, nonadecanol, eikosanol, heneikosanol, docosanol, tricosanol, tetracosanol, pentacosanol, hexacosanol, heptacosanol, octacosanol, nonacosanol, and triacosanol; branched alcohols such as isodecanol, isododecanol, isotetradecanol, isohexadecanol, isooctadecanol, isoeicosanol, isohenicosanol, isodocosanol, isotetracosanol, isohexacosanol, isooctacosanol, and isotriacosanol; linear alkenols such as hexenol, heptenol, octenol, nonenol, decenol, undecenol, dodecenol, tridecenol, tetradecenol, pentadecenol, hexadesenol, pentadecenol, hexadesenol, heptadesenol, octadesenol, nonadesenol, eisenol, docosenol, tetracosenol, pentacosenol, hexacosenol, heptacosenol, octacosenol, nonacosenol, and triaconsenol; and branched alkenols such as isohexenol, 2-ethylhexenol, isotridecenol, 1-methylheptadesenol, 1-hexylheptenol, isotridecenol, and isooctadesenol. Specific examples of metal soaps (e) include metal salts (saponified products) of fatty acids such as stearic acid, palmitic acid, myristic acid, aicosanoic acid, docosanoic acid, lauric acid, 12-hydroxystearic acid, araquinic acid, behenic acid, octanoic acid, and tall oil fatty acids, as well as resin acids such as abietic acid, neo-avietic acid, d-pimalic acid, iso-d-pimalic acid, podocalpic acid, agatendicarboxylic acid, benzoic acid, silicic acid, p-oxycytic acid, and diterponic acid. The types of metals used to constitute these metal salts are preferably metals other than alkali metals. Examples include aluminum, calcium, zinc, magnesium, silver, barium, beryllium, cadmium, cobalt, chromium, copper, iron, mercury, manganese, nickel, lead, tin, and titanium. Use of magnesium stearate and calcium stearate as metal soaps (e) is especially preferred. From the standpoint of handling and preventing precipitation in the treatment agent, the metal soap (e) is preferably a fine powder with an average particle diameter of 0.1 to 1.0 μm. The amount of silicone oil (c) and metal soap (e) used is preferably determined based on the intended use.

The following is a description of elastic fibers according to the present invention (elastic fibers of the present invention below). Elastic fibers of the present invention are elastic fibers to which a treatment agent of the present invention described above has been applied. There are no particular restrictions on the amount of treatment agent of the present invention applied to the elastic fibers, but application at a ratio of 0.1 to 10% by mass is preferred and at a ratio of 0.1 to 3% by mass is especially preferred.

Examples of elastic fibers include polyester-based elastic fibers, polyamide-based elastic fibers, polyolefin-based elastic fibers, and polyurethane-based elastic fibers. Among these, polyurethane-based elastic fibers are preferred.

The following is a detailed description of a method for manufacturing the polyurethane-based elastic fibers preferred as elastic fibers of the present invention. In the present invention, the method used to produce a spinning solution containing polyurethane (sometimes referred to as the “polyurethane spinning solution” below) or method used to produce the polyurethane solute of the solution may be the melt polymerization method or the solution polymerization method, but some other method may also be used. However, the solution polymerization is preferred. When the solution polymerization method is used, very few foreign substances such as gels are produced in the polyurethane, spinning is easy, and fine polyurethane elastic fibers are easy to obtain. When solution polymerization is used, the operation of making a solution can be omitted. This is an obvious advantage.

In an example of a polyurethane especially suitable for the present invention, polytetramethylene glycol (PTMG) with a molecular weight of 1500 or more and 6000 or less is used as the polymer diol, diphenylmethane diisocyanate (MDI) is used as the diisocyanate, and a diamine and/or diol is used as the chain extender. For example, a diamine such as ethylenediamine, 1,3-cyclohexanediamine, or 1,4-cyclohexanediamine is preferably used as the chain extender to form polyurethane urea. Ethylene glycol, 1,3-propanediol, 1,4-butanediol, neopentyl glycol, 1,2-propylene glycol, 1,4-cyclohexanedimethanol, 1,4-cyclohexanediol, 1,4-bis (β-hydroxyethoxy) benzene, bis (β-hydroxyethyl) terephthalate, or paraxylylenediol is preferably used as the diol. The chain extender is not limited to one type of diamine and/or diol. Multiple types of diamines and/or diols can be used. The melting point on the high temperature side of the yarn formed from polyurethane is preferably in the range of 200° C. or more and 280° C. or less.

A polyurethane can be synthesized using these raw materials in a solvent whose main component is DMAc, DMF, dimethyl sulfoxide (DMSO), and/or N-methyl-2-pyrrolidone (NMP). The so-called one-shot method can be used in which the raw materials are added to and dissolved in the solvent, and then heated to the appropriate temperature and reacted to obtain a polyurethane. Alternatively, a method can be used in which a polymer diol and diisocyanate are melted and reacted, and the reaction product is dissolved in a solvent and reacted with a diamine and/or diol mentioned above to obtain a polyurethane. These methods are especially preferred.

Typically, the melting point of the polyurethane on the high end is adjusted to a range from 200° C. to 280° C. by controlling the types and ratios of polymer diols, MDIs, diamines and/or diols. When the molecular weight of the polymer diol is low, a polyurethane having a melting point at a high temperature can be obtained by increasing the relative proportion of MDI. Similarly, when the molecular weight of the diamine and/or diol is low, a polyurethane having a melting point at a high temperature can be obtained by decreasing the relative proportion of polymer diol.

When the molecular weight of the polymer diol is 1800 or more, it is preferable to proceed with polymerization at a ratio of (number of moles of MDI)/(number of moles of polymer diol) of 1.5 or more in order to raise the melting point on the high end to 200° C. or more.

In addition, one or more end-sealing agents are preferably used in an elastic fiber of the present invention. Preferred examples of end-sealing agents include monoamines such as dimethylamine, diisopropylamine, ethylmethylamine, diethylamine, methylpropylamine, isopropylmethylamine, diisopropylamine, butylmethylamine, isobutylmethylamine, isopentylmethylamine, dibutylamine, and diamilamine; mono-ols such as ethanol, propanol, butanol, isopropanol, allyl alcohols, and cyclopentanol; and monoisocyanates such as phenylisocyanate.

A polyurethane elastic fiber of the present invention may also contain stabilizers, pigments and other additives. Examples include hindered phenolic agents such as BHT and Sumilyzer GA-80 from Sumitomo Chemical, benzotriazole-based and benzophenone-based agents such as Tinuvin from Ciba Geigy, phosphorus-based agents such as Sumilyzer P-16 from Sumitomo Chemical, and hindered amine agents serving as lightfasteners and antioxidants, etc.; pigments such as iron oxide and titanium oxide; inorganic materials such as zinc oxide, cerium oxide, magnesium oxide, calcium carbonate, and carbon black; fluorine-based or silicone-based resin powders; metallic soaps such as magnesium stearate; fungicides containing silver, zinc or compounds thereof; deodorants; and antistatic agents such as barium sulfate, cerium oxide, betaine and phosphoric acid-based agents. These are preferably included or reacted with the polymer. In order to further improve durability with respect to light and nitric oxide, nitric oxide supplements such as HN-150 from Japan Hydrazine, thermal oxidation stabilizers such as Sumilyzer GA-80 from Sumitomo Chemical, and light stabilizers such as Sumisorb 300 #622 from Sumitomo Chemical are preferably used.

The concentration of the resulting polyurethane spinning solution is preferably in the range of 30% by mass or more and 80% by mass or less.

The polyurethane elastic fiber of the present invention can be obtained by, for example, dry spinning, wet spinning, or melt spinning the spinning solution and then taking up the resulting fibers. Among these methods, dry spinning is preferred from the standpoint of being able to stably spin fibers of all finenesses, from thin to thick.

There are no particular restrictions on the fineness and cross-sectional profile of polyurethane elastic fibers of the present invention. For example, the cross-sectional profile of the yarn may be round or flat. There are no particular restrictions on the dry spinning method. Spinning conditions can be selected and spinning performed based on the desired properties and the spinning equipment being used.

For example, because the permanent strain rate and stress relaxation of a polyurethane elastic fiber of the present invention are strongly influenced by the speed ratio between the godet roller and the winding device, this is preferably set based on the intended use of the yarn. From the standpoint of a polyurethane elastic fiber with the desired permanent strain rate and stress relaxation, the speed ratio between the godet roller and the winding device is preferably set in the range between 1.10 and 1.65. When a polyurethane elastic fiber with an especially low permanent strain rate and stress relaxation is to be obtained, the speed ratio between the godet roller and the winding device is preferably in the range between 1.15 and 1.4, and more preferably in the range between 1.15 and 1.35. When a polyurethane elastic fiber with a high permanent strain rate and stress relaxation is to be obtained, the speed ratio between the godet roller and the winding device is preferably in the range between 1.25 and 1.65, and more preferably in the range between 1.35 and 1.65.

From the standpoint of improving the strength of the resulting polyurethane elastic fiber, the spinning speed is preferably 300 m/min or more.

Neat supplying is performed to attach the treatment agent of the present invention to elastic fibers in which the treatment agent is supplied without being diluted with a solvent or other component. The attachment step can be performed after spinning and before winding into a package, when the taken-up package is unwound, or during warping with a warping machine. In any of these steps, the attachment method can be any method common in the art, such as the roller supply method, guide supply method, or the spray supply method. The amount of treatment agent attached is from 0.1 to 5% by mass relative to the elastic fiber. However, the amount attached is preferably from 0.1 to 3% by mass from the standpoint of achieving a good balance between hot melt adhesiveness and unraveling properties. The treatment agent of the present invention is preferably applied as a spinning oil agent immediately after the elastic fiber has been spun.

The hot melt adhesive is preferably one that adheres in the temperature range from 120° C. to 180° C. Examples of polymer materials in the hot melt adhesive include hydrogenated SBS (styrene-butadiene-styrene block) copolymer, ethylene vinyl acetate (EVA), polyolefin copolymers, synthetic rubber-based hot melt materials, polyamide-based hot melt materials, polyester-based hot melt materials, and polyurethane-based hot melt materials.

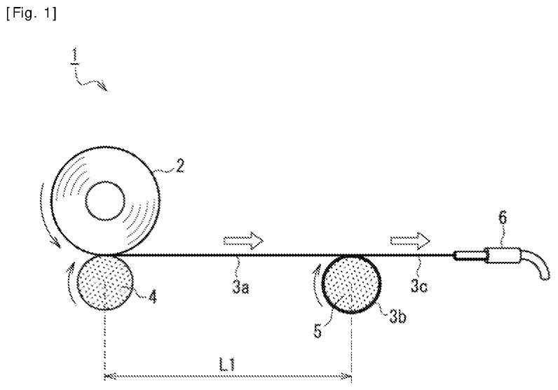

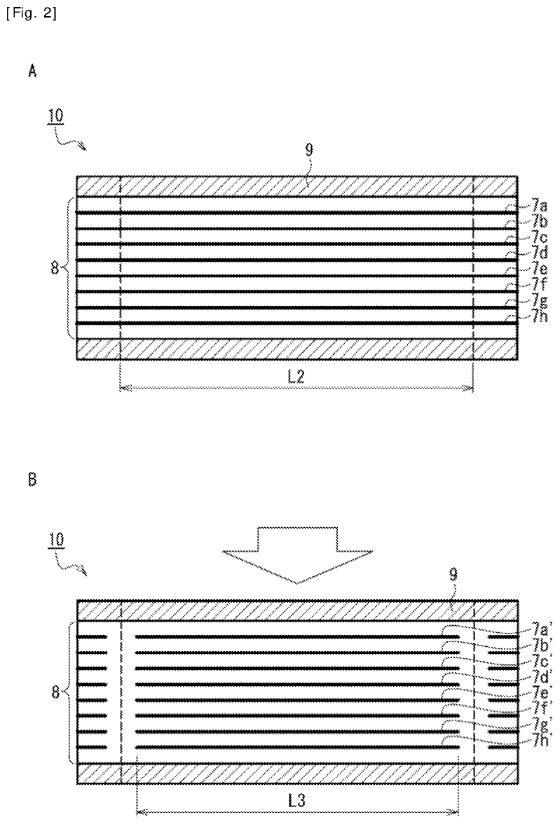

FIG. 1 is a schematic diagram used to explain the elastic fiber unraveling stability tester used in the examples of the present invention. FIG. 2A and FIG. 2B are schematic diagrams used to explain the hot melt adhesiveness testing method in the same. A detailed description follows in the examples.

### EXAMPLES

The following is a more detailed description of the present invention with reference to examples. However, the present invention is not limited to these embodiments. First, the methods used to evaluate the various properties in the present invention will be explained.

### [Kinematic Viscosity of the Treatment Agent]

The kinematic viscosity at 30° C. was measured by the Canon-Fenske method (unit: mm²/s).

### [Swelling Rate of the Treatment Agent]

After cutting commercially available polyurethane film into 6 cm×10 cm sections and weighing them precisely, they were immersed for 7 minutes in various oil agents. After wiping off the oil agent adhering to the surface, the weight was measured and the weight increase rate in the film was used as the swelling rate.

### [Stability of the Treatment Agent]

The prepared elastic fiber treatment agent was allowed to stand at 25° C. for 3 months, and the stability was evaluated according to the following criteria.

A (Excellent): There was no precipitation or separation, and the uniform state at the time of preparation was maintained.

B (Good): Some precipitation or separation occurred, but the uniform state at the time of preparation was restored by stirring.

C (Poor): Precipitation and separation occurred, and the uniform state at the time of preparation was not restored by stirring.

### [Unraveling Stability Testing on the Polyurethane Elastic Fiber]

After leaving 4.5 kg wound yarn of a polyurethane elastic fiber in an atmosphere of 35° C. and 65% RH for 14 days, the wound yarn was unwound from the winding paper tube to 1 cm, and the unwound yarn was tested using the unraveling stability tester shown in FIG. 1. This unraveling stability tester 1 has a winding 2, textured rollers 4, 5, and an aspirator 6. The surface of the winding 2 is placed in contact with the textured roller 4. While rotating the textured roller 4, the polyurethane elastic fiber 3a was discharged at a constant surface speed for the textured roller 4 of 30 m/min (S1). The discharged polyurethane elastic fiber 3a was run around a textured roller 5 of the same diameter installed at a distance of L1=100 cm (polyurethane elastic fiber 3b). When the surface speed of the textured roller 5 was gradually changed and the polyurethane elastic fiber 3a was separated from the textured roller 4, the minimum speed (S2) of the textured roller 5 was determined at which the winding 2 of the polyurethane elastic fiber 3a was discharged smoothly without lifting. The speed ratio (S2)/(S1) of both textured rollers 4, 5 was used to define the unraveling properties of polyurethane elastic fiber 3a. The portion 1 cm from the outside of the 4.5 kg winding was measured as the unraveling properties (A) of the outer layer, the portion 1 cm from the inside was measured as the unraveling properties (B) of the inner layer, and the unraveling stability (B)−(A) due to the winding layers was determined. The polyurethane elastic fiber 3c passing through the textured roller 5 was suctioned using the aspirator 6. A low value for the unraveling stability (B)−(A) indicates stable separation of polyurethane elastic fibers between layers. The unraveling properties test was performed using two windings and the average value was used in the evaluation.

### [Hot Melt Adhesiveness Test]

While running polyurethane elastic fibers at a speed of 130 m/min and stretching a polypropylene non-woven fabric with a width of 15 cm at the specified draft (draft 3.0), eight fibers were run at equal intervals in the same direction, a hot melt adhesive containing an SBS (styrene-butadiene-styrene block) copolymer dissolved in a pot at 150° C. as the main component was applied with a comb gun to a specified amount (0.05 g/m) per polyurethane elastic fiber. Afterwards, another thin, transparent polypropylene non-woven fabric was placed on top and pressure-bonded, and an elastic sheet taken up. As shown in FIG. 2A and FIG. 2B, the elastic sheet 8 was fixed to a flat wooden plate 9 with the non-woven fabric (not shown) completely stretched. From the top of the elastic sheet 8, a razor blade was used to cut eight polyurethane elastic fibers 7a-7h with a length of L2=30 cm from the non-woven fabric at both ends for a total of 16 locations. The stretching plate 10 was stored at 40° C. and 80% RH to cause the polyurethane elastic fibers 7a-7h fixed with hot melt adhesive to shrink, that is, slip in polypropylene non-woven fabric. Afterwards, the length (L3) of the fibers 7a′-7h′ was measured and the length (L2) between the two cut portions (L2) was measured as the original length. Measurements were taken during storage after two hours and after eight hours. The measurements were performed on a total of 24 elastic fibers, and the average values of the hot melt adhesive retention rates for these 24 fibers were evaluated.

Hot melt adhesiveness retention rate(%)=100×(L3)/(L2)

A higher hot melt adhesiveness retention rate is better.

### [Preparation of the Treating Agents]

Each component was blended at the composition ratios for A1 to A10 and for B1 to B6 in Table 1. At this time, the treatment agents containing hydrocarbon resins were prepared by stirring at 40° C. until the components were completely dissolved. The treatment agents containing metal soap components were prepared by dispersing the components using a ball mill.

### [Hydrocarbon Resin (A) Details]

The following hydrocarbon resins (A) were used.

a-1: Fully hydrogenated aromatic petroleum hydrocarbon resin with structural components including indene and methylstyrene serving as starting materials: softening point 90° C.

a-2: Fully hydrogenated petroleum hydrocarbon resin of copolymerized petroleum resin of aromatic components and aromatic components with structural components including dicyclopentadiene, indene, and methylstyrene serving as starting materials: softening point 99° C.

a-3: Partially hydrogenated aromatic petroleum hydrocarbon resin with structural components including indene and methylstyrene serving as starting materials: softening point 135° C.

### [Hydrocarbon Oil (B) Details]

Liquid paraffin was used as the hydrocarbon oil (B), and the number of seconds required for 50 ml of the sample to flow down was measured at 40° C. using Redwood Viscometer No. 827 from Yoshida Seisakusho Co., Ltd.

### [Silicone Oil (c) Details]

Polydimethylsiloxane was used with a kinematic viscosity of 20×10⁻⁶ m²/s at 25° C. as measured with a Canon-Fenske viscometer in accordance with NS Z8803-2011.

### [Higher Alcohol (d) Details]

Isohexadecanol was used.

### [Metal Soap (e) Details]

Magnesium stearate was used, and the treatment agent was prepared for use by wet pulverization so that the average particle size of the magnesium stearate was 0.4 to 0.6 μm. The average particle size was determined by using a laser diffraction/scattering type particle size distribution measuring device, and the number-based median diameter was used as the average particle size.

TABLE 1

| | | Hydrocarbon Resin (A) | | | Hydrocarbon Oil (B) | | |
|---|---|---|---|---|---|---|---|
| | Treatment | Softening | Softening | Softening | Visc. | Visc. | Visc. |
| | Agent | Pt. 90° C. | Pt. 99° C. | Pt. 135° C. | 200 sec | 100 sec | 80 sec |
| | Name | a-1 | a-2 | a-3 | (40° C.) | (40° C.) | (40° C.) |
| Ex. 1 | A1 | 5 | 0 | 0 | 0 | 95 | 0 |
| Ex. 2 | A2 | 5 | 0 | 0 | 0 | 0 | 95 |
| Ex. 3 | A3 | 0 | 5 | 0 | 0 | 95 | 0 |
| Ex. 4 | A4 | 0 | 0 | 5 | 0 | 95 | 0 |
| Ex. 5 | A5 | 10 | 0 | 0 | 0 | 0 | 90 |
| Ex. 6 | A6 | 10 | 0 | 0 | 0 | 0 | 75 |
| Ex. 7 | A7 | 0 | 0 | 10 | 0 | 59 | 0 |
| Ex. 8 | A8 | 20 | 0 | 0 | 0 | 0 | 70 |
| Ex. 9 | A9 | 0 | 5 | 0 | 0 | 0 | 94 |
| Ex. 10 | A10 | 5 | 0 | 0 | 0 | 65 | 0 |
| C. Ex. 1 | B1 | 0 | 0 | 0 | 0 | 100 | 0 |
| C. Ex. 2 | B2 | 0 | 0 | 0 | 0 | 0 | 100 |
| C. Ex. 3 | B3 | 0 | 0 | 0 | 0 | 0 | 85 |
| C. Ex. 4 | B4 | 0 | 0 | 0 | 0 | 70 | 0 |
| C. Ex. 5 | B5 | 0 | 0 | 0 | 0 | 69 | 0 |
| C. Ex. 6 | B6 | 0 | 0 | 0 | 85 | 0 | 0 |

| | Silicone | Higher | Metal | | | |
|---|---|---|---|---|---|---|
| | Oil (c) | Alcohol (d) | Soap (e) | Evaluation of Treatment Agent | | |
| | Amt. in | Amt. in | Amt. in | Kinematic | | |
| | Treatment | Treatment | Treatment | Viscosity | Swelling | |
| | Agent | Agent | Agent | 30° C. | Rate | Stability |
| | (mass %) | (mass %) | (mass %) | mm²/s | (%) | — |
| Ex. 1 | 0 | 0 | 0 | 21 | 2.0 | A |
| Ex. 2 | 0 | 0 | 0 | 18 | 2.1 | A |
| Ex. 3 | 0 | 0 | 0 | 22 | 2.0 | A |
| Ex. 4 | 0 | 0 | 0 | 23 | 2.0 | A |
| Ex. 5 | 0 | 0 | 0 | 23 | 1.9 | A |
| Ex. 6 | 15 | 0 | 0 | 19 | 1.8 | A |
| Ex. 7 | 15 | 15 | 1 | 27 | 2.0 | B |
| Ex. 8 | 5 | 5 | 0 | 31 | 1.9 | A |
| Ex. 9 | 0 | 0 | 1 | 22 | 1.6 | B |
| Ex. 10 | 15 | 15 | 0 | 23 | 1.9 | A |
| C. Ex. 1 | 0 | 0 | 0 | 18 | 3.0 | A |
| C. Ex. 2 | 0 | 0 | 0 | 15 | 2.9 | A |
| C. Ex. 3 | 15 | 0 | 0 | 13 | 2.8 | A |
| C. Ex. 4 | 15 | 15 | 0 | 17 | 2.7 | A |
| C. Ex. 5 | 15 | 15 | 1 | 17 | 2.6 | B |
| C. Ex. 6 | 15 | 0 | 0 | 26 | 3.4 | A |

### Example 1

MDI and PTMG having a number average molecular weight of 1800 were placed in a container at a molar ratio of MDI/PTMG=1.58/1, components were reacted at 90° C., and the reaction product was dissolved in N,N-dimethylacetamide (DMAc). Next, a DMAc solution containing ethylenediamine and diethylamine was added to the solution in which the reaction product was dissolved to prepare a polyurethane urea solution having a polymer solid content of 35% by mass. A condensed polymer of p-cresol and divinylbenzene (Metachlor® 2390 from DuPont) serving as the antioxidant and 2-[4,6-bis (2,4-dimethylphenyl)-1,3,5-triazine-2-yl]-5-(octyloxy) phenol (Cyasorb® 1164 from Solvay) serving as the UV absorber were mixed in at 3:2 (mass ratio) to adjust the DMAc solution (concentration 35% by mass). This was used as an additive solution (35% by mass). The polyurethane urea solution and the additive solution were mixed together at a ratio of 98% by mass and 2% by mass to prepare a polyurethane spinning solution (X1). This spinning solution (Y1) was dry-spun at a winding speed of 500 m/min, and 1.5 parts by mass of treatment agent A1 was applied to 100 parts by mass of polyurethane elastic fiber during winding to prepare polyurethane elastic fibers (580 decitex, 56 filaments) and obtain 4.5 kg wound yarn.

### Examples 2-10, Comparative Examples 1-6

As shown in Table 1, polyurethane elastic fiber 4.5 kg wound yarn was obtained in the same manner as in Example 1 except that the type of treatment agent was changed. The results from evaluating the resulting yarn are shown in Table 2. The polyurethane elastic fibers in Examples 1 to 10 had sufficient performance in all of the evaluations. In Comparative Examples 1 to 6, by contrast, the results were not satisfactory for either unraveling stability or hot melt adhesiveness.

TABLE 2

| | | Elastic Fibers with 1.5% by mass Treatment Agent Applied | | | | |
|---|---|---|---|---|---|---|
| | | (A) | (B) | | Hot Melt Adhesiveness | |
| | Name of | Dissolvability | Dissolvability | | Retention Rate (%) | |
| | Treatment | Outer Layer | Inner Layer | (B) − (A) | After | After |
| | Agent | (—) | (—) | (—) | 2 Hrs | 8 Hrs |
| Ex. 1 | A1 | 1.40 | 2.01 | 0.61 | 94 | 92 |
| Ex. 2 | A2 | 1.31 | 2.04 | 0.73 | 96 | 93 |
| Ex. 3 | A3 | 1.38 | 2.00 | 0.62 | 93 | 92 |
| Ex. 4 | A4 | 1.35 | 1.99 | 0.64 | 95 | 92 |
| Ex. 5 | A5 | 1.42 | 1.97 | 0.55 | 96 | 94 |
| Ex. 6 | A6 | 1.31 | 1.93 | 0.62 | 94 | 90 |
| Ex. 7 | A7 | 1.43 | 1.99 | 0.56 | 95 | 89 |
| Ex. 8 | A8 | 1.33 | 1.95 | 0.62 | 96 | 94 |
| Ex. 9 | A9 | 1.24 | 1.83 | 0.59 | 93 | 90 |
| Ex. 10 | A10 | 1.27 | 1.98 | 0.71 | 94 | 91 |
| C. Ex. 1 | B1 | 1.21 | 2.30 | 1.09 | 91 | 83 |
| C. Ex. 2 | B2 | 1.19 | 2.27 | 1.08 | 91 | 84 |
| C. Ex. 3 | B3 | 1.07 | 2.23 | 1.16 | 89 | 76 |
| C. Ex. 4 | B4 | 1.19 | 2.21 | 1.02 | 88 | 77 |
| C. Ex. 5 | B5 | 1.15 | 2.16 | 1.01 | 88 | 74 |
| C. Ex. 6 | B6 | 1.42 | 2.46 | 1.04 | 78 | 65 |

### INDUSTRIAL APPLICABILITY

Because an elastic fiber treatment agent of the present invention imparts excellent unraveling properties to elastic fibers and imparts excellent adhesiveness to hot melt adhesives, it is suitable for use in sanitary products with excellent comfort and fit, such as disposable diapers and sanitary napkins.

### KEY TO THE DRAWINGS

1: Unraveling stability tester
2: Winding
3a, 3b, 3c: Polyurethane elastic fiber
4, 5: Textured roller
6: Aspirator
7a-7h: Polyurethane elastic fiber before shrinkage
7a′-7h′: Polyurethane elastic fiber after shrinkage
8: Elastic sheet
9: Flat wooden plate

1. An elastic fiber having an elastic fiber treatment agent attached to the fiber surface, comprising: a hydrocarbon resin (A) having a structure in which a polymer including a structural unit whose monomer is at least one selected from aromatic olefins and aliphatic diolefins as the main structural unit is partially or fully hydrogenated; and a hydrocarbon oil (B).

2. An elastic fiber according to claim 1, wherein the partial hydrogenation is a polymer in which at least 50% of the double bonds and less than 100% of the double bonds in the polymer are hydrogenated.

3. An elastic fiber according to claim 1, wherein the aromatic olefin is at least one selected from indene and methylstyrene.

4. An elastic fiber according to claim 1, wherein the aliphatic diolefin is isoprene.

5. An elastic fiber according to claim 1, wherein the softening point of the hydrocarbon resin (A) is 70° C. or higher and 140° C. or lower.

6. An elastic fiber according to claim 1, wherein the elastic fiber comprises 0.1% by mass or more and 40% by mass or less of hydrocarbon resin (A) when the treatment agent is used as a parameter.

7. An elastic fiber according to claim 1, wherein 10% by mass or more of the hydrocarbon resin (A) dissolves in the hydrocarbon oil (B) at 20° C.

8. An elastic fiber according to claim 1, wherein the hydrocarbon resin (A) is insoluble in N,N-dimethylacetamide (DMAc) and/or N,N-dimethylformamide (DMF).

9. An elastic fiber according to claim 1, wherein the hydrocarbon oil (B) is a mineral oil.

10. An elastic fiber according to claim 1, wherein from 0.1 to 10% by mass of the N,N elastic fiber treatment agent adheres to the elastic fiber.

11. An elastic fiber according to claim 1, wherein the swelling rate of polyurethane is 2.2% or less when the treatment agent is attached to the polyurethane.

12. A treatment agent for an elastic fiber according claim 1, wherein the treatment agent is a spinning oil agent.

13. A fiber structure comprising an elastic fiber according to claim 1.

14. A fiber structure according to claim 13, wherein the fiber structure is a sanitary product.
